(19) United States
(12) Patent Application Publication
(10) Pub. No.: US 2007/0190000 A1
(43) Pub. Date: Aug. 16, 2007

(54) Matting cosmetic composition

(75) Inventors: Pascal Arnaud, I'Hay les Roses (FR); Nathalie Gavache, Orleans (FR)

Correspondence Address: OLIFF & BERRIDGE, PLC, P.O. BOX 19928, ALEXANDRIA, VA, 22320, US

(73) Assignee: L'OREAL, PARIS (FR)

(21) Appl. No.: 11/698,199

(22) Filed: Jan. 26, 2007

Related U.S. Application Data
(60) Provisional application No. 60/780,057, filed on Mar. 8, 2006.

(30) Foreign Application Priority Data
Jan. 26, 2006 (FR) 06 50282

(51) Int. Cl. A61K 8/18; A61Q 1/02; A61Q 1/04; A61Q 1/08

(52) U.S. Cl. 424/63; 424/78.03

(57) ABSTRACT

The present invention relates to a non-compacted cosmetic composition for caring for and/or making up keratin fibres, comprising, in a physiologically acceptable medium, at least 22% by weight of water relative to the total weight of the composition and at least one fatty phase comprising at least one dispersion of micronized wax particles, the said particles being present in a content of between 5% and 50% by weight relative to the total weight of the composition.

This non provisional application claims the benefit of French Application No. 06 50282 filed on Jan. 26, 2006 and U.S. Provisional Application No. 60/780,057 filed on Mar. 8, 2006.

The present invention relates to a cosmetic composition for making up and/or caring for keratin materials, for example of the face, for example the skin and/or the lips.

For example, the compositions under consideration according to the invention may constitute a makeup product, for example for the face, which allows a matting effect to be obtained on the skin while at the same time maintaining a natural appearance.

By definition, a matting product is a product that prevents the skin from shining.

Compositions with a matting effect are already known. However, they are not entirely satisfactory.

Products for making up and/or caring for human skin and/or lips, for instance foundations or lipsticks, generally contain a fatty phase based on oil(s) and/or wax(es), pigments and/or fillers and optionally additives, for instance cosmetic or dermatological active agents.

It is thus known practice to use fillers to obtain a matting effect.

These fillers are usually chosen as a function of their satisfactory oil-absorbing properties and/or their capacity to scatter light efficiently.

However, fillers may have the drawback of giving the compositions in which they are formulated a powdery appearance that is detrimental to a natural appearance, after applying the said composition to a support.

Moreover, such compositions may have a heavy and dry texture that is detrimental as regards the feel and the application.

There is thus a need for a fluid cosmetic composition that is pleasant to apply, which can give matting makeup and/or care that does not have a powdery effect after application.

The inventors have found, unexpectedly, that it is possible to overcome the abovementioned drawback by incorporating, into the fatty phase of a care and/or makeup composition, an effective amount of wax particles in micronized form, while at the same time maintaining a minimum water content sufficient to ensure good cosmetic properties.

Consequently, according to one exemplary embodiment, the present invention relates to a non-compacted cosmetic composition for caring for and/or making up keratin materials, comprising, in a physiologically acceptable medium, at least 22% by weight of water relative to the total weight of the composition and at least one fatty phase comprising at least one dispersion of micronized wax particles, the said particles being present in a content of between 5% and 50% by weight relative to the total weight of the composition.

The compositions according to the invention may be for example in a fluid form.

This composition may for example be in the form of a paste or a fluid cream, for instance foundations, skin makeup bases or fluid lipsticks, eyeliners, antisun compositions, skin colouring compositions or body makeup compositions.

According to one exemplary embodiment, the compositions according to the invention are in the form of a fluid foundation or a fluid skin makeup base.

For the purposes of the present invention, the term “fluid” denotes a composition capable of flowing under the action of its own weight, at room temperature (25° C.), as opposed to compounds of paste type.

In general, the compositions according to the invention have a physiologically acceptable medium.

The term “keratin materials” is intended to cover the skin, mucous membranes, for instance the lips, the nails and keratin fibres, such as the eyelashes and the hair. The cosmetic compositions in accordance with the present invention may for example be advantageous for application to the skin and/or the lips.

The terms “between . . . and . . . ” and “ranging from . . . to . . . ” mean that the limits are also included.

Micronized Wax Particles

In general, the term “wax” means a lipophilic compound, which is solid at room temperature (25° C.), with a reversible solid/liquid change of state, and which has a melting point of greater than or equal to 60° C. that may be up to 160° C., or even 200° C.

The wax particles constituting one of the characteristics of the present invention are in a micronized form.

For the purposes of the present invention, the terms “micronized wax” and “microwax” mean a wax in the form of a powder whose particles have a number-average size of less than or equal to 50 μm, for example ranging from 0.5 to 50 μm, for example ranging from 1 to 30 μm or for example ranging from 3 to 20 μm.

The term “number-average size” denotes the dimension given by the statistical particle size distribution to half the population, written as D50.

The microwaxes that may be used in the compositions according to the invention are chosen from waxes that are solid and rigid at room temperature, of animal, plant, mineral or synthetic origin, and mixtures thereof.

The microwaxes according to the invention may for example have a melting point ranging from 60° C. to 160° C., for example greater than or equal to 80° C., for example ranging from 80° C. to 150° C., or for example greater than or equal to 100° C., for example ranging from 100° C. to 140° C.

The melting point of the microwax may be measured using a differential scanning calorimeter (D.S.C.), for example the calorimeter sold under the name DSC 30 by the company Mettler. A 15 mg sample of product placed in a crucible is subjected to a first temperature rise ranging from 0° C. to 160° C., at a heating rate of 10° C./minute, and is then cooled from 160° C. to 0° C. at a cooling rate of 10° C./minute and is finally subjected to a second temperature rise ranging from 0° C. to 160° C. at a heating rate of 5° C./minute. During the second temperature rise, the variation in the difference of power absorbed by the empty crucible and by the crucible containing the sample of product is measured as a function of the temperature. The melting point of the compound is the value of the temperature corresponding to the top of the peak of the curve representing the variation of the difference in power absorbed as a function of the temperature.

It should be noted that, in the manufacturing process, care will be taken not to melt the microwax. Advantageously, the temperature of the composition during the manufacturing process does not exceed a temperature 30° C. below the melting point of the microwax.

The micronized waxes used in the present application may be chosen from hydrocarbon-based, silicone and/or fluoro micronized waxes, optionally comprising ester, amide, acid or hydroxyl functions.

They may for example be of natural origin and may be chosen from optionally modified beeswax, carnauba wax, candelilla wax, paraffin waxes, microcristalline waxes, montan wax, hydrogenated oils, for instance hydrogenated jojoba oil or hydrogenated castor oil, or waxes resulting from the polymerization of ethylene and/or propylene, waxes obtained by Fischer-Tropsch synthesis, fatty acid esters and amides, silicone waxes, for instance alkyl-, alkoxy- and/or esters of poly(di)methylsiloxane that are solid at room temperature, containing from 10 to 45 carbon atoms, and fatty acids, for instance stearic acid or behenic acid, and mixtures thereof.

As microwaxes that may be used in the compositions according to the invention, mention may be made for example of carnauba microwaxes such as those sold under the name MicroCare 350 by the company Micro Powders, microwaxes of synthetic wax such as those sold under the name MicroEase 114S® by the company Micro Powders, microwaxes consisting of a mixture of carnauba wax and of polyethylene wax, such as those sold under the names MicroCare 300® and 310® by the company Micro Powders, microwaxes consisting of a mixture of carnauba wax and of synthetic wax, such as those sold under the name MicroCare 325® by the company Micro Powders, polyethylene microwaxes such as those sold under the names MicroPoly 200®, 220®, 220L® and 250S® by the company Micro Powders, and those sold under the name Cerapure H5-C by the company Shamrock, or polypropylene microwaxes such as those sold under the name Mattewax by the company Micro Powders.

The micronized wax particles may be present in a composition according to the invention in a content of between 5% and 50% by weight, for example between 10% and 30% by weight or for example between 15% and 25% by weight, relative to the total weight of the composition.

Aqueous Phase

The composition according to the invention also comprises at least one aqueous medium, constituting an aqueous phase, which may form the dispersed phase or the continuous phase of the composition.

The aqueous phase may consist exclusively of water.

The water may be a floral water such as cornflower water and/or a mineral water such as eau de Vittel, eau de Lucas or eau de La Roche Posay and/or a spring water.

The aqueous phase may also comprise a mixture of water and of water-miscible organic solvent(s) (miscibility with water of greater than 50% by weight at 25° C.), for instance monoalcohols containing from 1 to 5 carbon atoms such as ethanol and isopropanol, glycols containing from 2 to 8 carbon atoms such as glycerol, propylene glycol, 1,3-butylene glycol, dipropylene glycol or diethylene glycol, glycol ethers such as mono-, di- or tripropylene glycol (C₁-C₄)alkyl ethers and mono-, di- or triethylene glycol (C₁-C₄)alkyl ethers, C₃-C₄ ketones and C₂-C₄ aldehydes, and mixtures thereof.

The aqueous phase (water and optionally the water-miscible organic solvent(s)) may also comprise stabilizers, for instance sodium chloride, magnesium dichloride and magnesium sulfate.

The aqueous phase may also comprise any water-soluble or water-dispersible compound that is compatible with an aqueous phase, such as care active agents, gelling agents, film-forming polymers, thickeners and surfactants, and mixtures thereof.

The compositions according to the invention comprise at least 22% by weight of water relative to the total weight of the composition. They may for example comprise at least 25% by weight of water relative to the total weight of the composition.

The aqueous phase, and for example the water, may be present in the composition according to the invention in a content ranging from 30% to 80% by weight and for example from 35% to 50% by weight relative to the total weight of the composition.

As mentioned previously, the compositions may for example be in a fluid form, i.e. different from solid forms of stick type, or compacted.

The composition may for example be in the form of an emulsion that may be simple, of the oil-in-water (O/W) or water-in-oil (W/O) type or a multiple emulsion (for example water-oil-water or oil-water-oil) whose use is well known to those skilled in the art.

According to one exemplary embodiment of the invention, the composition is in the form of a water-in-oil emulsion.

It may also comprise a surfactant or a mixture of surfactants whose HLB (hydrophilic/lipophilic balance) is generally adapted to the nature of the emulsion to be stabilized.

As surfactants that may be used in the invention, suitable for obtaining a W/O emulsion, mention may be made of those with an HLB of less than 7 and for example fatty acid esters of polyols, for instance sorbitol or glyceryl mono-, di-, tri- or sesquioleates or stearates, and glyceryl or polyethylene glycol laureates; alkyl or alkoxy dimethicone copolyols whose alkyl or alkoxy chain is pendent or at the end of a silicone backbone, for example containing from 6 to 22 carbon atoms. As surfactants that may be used in the invention for obtaining an O/W emulsion, mention may be made of those with an HLB of greater than 7, for instance fatty acid esters of polyethylene glycol (polyethylene glycol monostearate or monolaurate); polyoxyethylenated fatty acid esters (stearate, oleate) of sorbitol; polyoxyethylenated alkyl (lauryl, cetyl, stearyl, octyl) ethers and dimethicone copolyols. In general, any ionic (cationic or anionic) amphoteric surfactant and any non-ionic surfactant that are well known to those skilled in the art may be used.

The surfactant may be present in the composition in a content ranging from 0.3% to 10% by weight and for example from 1% to 5% by weight relative to the total weight of the composition.

Fatty Phase

The cosmetic compositions in accordance with the present invention also comprise a fatty phase comprising, besides the microwaxes under consideration according to the invention, for example at least one compound chosen from oils and fatty substances that are solid at room temperature (20-25° C.) and atmospheric pressure, for instance non-micronized waxes and pasty fatty substances, and mixtures thereof.

The fatty phase may be advantageously present in a relative ratio that is sufficient to allow the micronized wax particles under consideration according to the invention to be really dispersed therein.

Thus, the fatty phase may be generally present in a total weight content that is greater than or equal to the total weight content of micronized wax particles.

In particular, the total weight content of oils may be greater than or equal to the total weight content of micronized wax particles.

a) Oil

The term “oil” means any fatty substance that is in liquid form at room temperature (20-25° C.) and at atmospheric pressure. The liquid fatty phase may also contain, besides oils, other compounds dissolved in the oils, such as gelling agents and/or structuring agents.

The cosmetic composition according to the present invention may comprise at least one and for example at least two oils.

The oils that are suitable for preparing the cosmetic compositions according to the invention may be volatile or non-volatile oils.

For the purposes of the present invention, the term “volatile oil” means an oil (or non-aqueous medium) that is capable of evaporating on contact with the skin in less than one hour, at room temperature and atmospheric pressure. The volatile oil is a volatile cosmetic oil, which is liquid at room temperature, for example having a non-zero vapour pressure, at room temperature and atmospheric pressure, for example having a vapour pressure ranging from 0.13 Pa to 40 000 Pa (10⁻³ to 300 mmHg), for example ranging from 1.3 Pa to 13 000 Pa (0.01 to 100 mmHg) and for example ranging from 1.3 Pa to 1300 Pa (0.01 to 10 mmHg).

For the purposes of the present invention, the term “non-volatile oil” means an oil with a vapour pressure of less than 0.13 Pa.

The volatile or non-volatile oils may be hydrocarbon-based oils for example of animal, plant or mineral origin, synthetic oils, silicone oils or fluoro oils, or mixtures thereof.

For the purposes of the present invention, the term “hydrocarbon-based oil” means an oil with a hydrocarbon-based backbone, i.e. mainly containing hydrogen and carbon atoms and possibly oxygen, nitrogen, sulfur and/or phosphorus atoms.

Non-volatile hydrocarbon-based oils that may for example be mentioned include:
synthetic ethers containing from 10 to 40 carbon atoms;
linear or branched hydrocarbons of mineral or synthetic origin, such as petroleum jelly, polydecenes, hydrogenated polyisobutene such as Parleam®, Squalane®, and mixtures thereof, and in particular hydrogenated polyisobutene, and

synthetic esters, for instance the oils of formula R₁COOR₂ in which R₁ represents a linear or branched fatty acid residue containing from 1 to 40 carbon atoms and R₂ represents a hydrocarbon-based chain, which is for example branched, containing from 1 to 40 carbon atoms, on condition that R₁+R₂≧10.

The esters may be chosen for example from esters, for example of fatty acids, for instance:
cetostearyl octanoate, isopropyl alcohol esters such as isopropyl myristate, isopropyl palmitate, ethyl palmitate, 2-ethylhexyl palmitate, isopropyl stearate or isostearate, isostearyl isostearate, octyl stearate, hydroxylated esters, for instance isostearyl lactacte, octyl hydroxystearate, diisopropyl adipate, heptanoates, and especially isostearyl heptanoate, alcohol or polyalcohol octanoates, decanoates or ricinoleates, for instance propylene glycol dioctanoate, cetyl octanoate, tridecyl octanoate, 2-ethylhexyl 4-diheptanoate and palmitate, alkyl benzoates, polyethylene glycol diheptanoate, propylene glycol diethyl 2-hexanoate and mixtures thereof, C₁₂ to C₁₅ alkyl benzoates, hexyl laurate, neopentanoic acid esters, for instance isodecyl neopentanoate, isotridecyl neopentanoate, isostearyl neopentanoate, octyldodecyl neopentanoate, isononanoic acid esters, for instance isononyl isononanoate, isotridecyl isononanoate, octyl isononanoate, and hydroxylated esters, for instance isostearyl lactate and diisostearyl malate;
polyol esters and pentaerythritol esters, for instance dipentaerythrityl tetrahydroxystearate/tetraisostearate,
esters of diol dimers and diacid dimers such as Lusplan DD-DA5® and Lusplan DD-DA7® sold by the company Nippon Fine Chemical and described in patent application FR 03/02809 filed on 6 Mar. 2003,
fatty alcohols that are liquid at room temperature, with a branched and/or unsaturated carbon-based chain containing from 12 to 26 carbon atoms, for instance 2-octyldodecanol, isostearyl alcohol, oleyl alcohol, 2-hexyldecanol, 2-butyloctanol and 2-undecylpentadecanol, and
dialkyl carbonates, the two alkyl chains possibly being identical or different, such as the dicaprylyl carbonate sold under the name Cetiol CC® by Cognis.

The volatile hydrocarbon-based oils may be chosen from hydrocarbon-based oils containing from 8 to 16 carbon atoms, and for example branched C₈-C₁₆ alkanes (also known as isoparaffins), for instance isododecane (also known as 2,2,4,4,6-pentamethylheptane), isodecane, isohexadecane and, for example, the oils sold under the trade names Isopar® and Permethyl®.

The compositions according to the invention may also contain volatile or non-volatile silicone oils.

The non-volatile silicone oils that may be used in the composition according to the invention may be non-volatile polydimethylsiloxanes (PDMS), polydimethylsiloxanes comprising alkyl or alkoxy groups that are pendent and/or at the end of a silicone chain, these groups each containing from 2 to 24 carbon atoms, phenyl silicones, for instance phenyl trimethicones, phenyl dimethicones, phenyl trimethylsiloxydiphenylsiloxanes, diphenyl dimethicones, diphenylmethyldiphenyltrisiloxanes, 2-phenylethyl trimethylsiloxysilicates, and dimethicones or phenyl trimethicone with a viscosity of less than or equal to 100 cSt, and mixtures thereof.

Volatile silicone oils that may more particularly be used include volatile linear or cyclic silicone oils, for example those with a viscosity ≦8 centistokes (8×10⁻⁶ m²/s) and for example containing from 2 to 10 and for example from 2 to 7 silicon atoms, these silicones optionally comprising alkyl or alkoxy groups containing from 1 to 10 carbon atoms. As volatile silicone oils that may be used in the invention, mention may be made for example of dimethicones with a viscosity of 5 and 6 cSt, octamethylcyclotetrasiloxane, decamethylcyclopentasiloxane, dodecamethylcyclohexasiloxane, heptamethylhexyltrisiloxane, heptamethyloctyltrisiloxane, hexamethyldisiloxane, octamethyltrisiloxane, decamethyltetrasiloxane and dodecamethylpentasiloxane, and mixtures thereof.

Volatile fluoro oils such as nonafluoromethoxybutane or perfluoromethylcyclopentane, and mixtures thereof, may also be used.

A composition according to the invention may also comprise one or more oil(s) in a content ranging from 10% to 70%, for example from 12% to 60% or for example from 15% to 50% by weight relative to the total weight of the composition.

The compositions according to the invention may also comprise for example at least one compound chosen from non-micronized waxes and pasty fatty substances, and mixtures thereof.

b) Non-Micronized Waxes

Besides micronized waxes, the compositions according to the invention may comprise one or more waxes chosen, for example, from synthetic waxes, for instance polyethylene wax (for example with a molecular weight of between 400 and 600) or Fischer-Tropsch waxes, silicone waxes, for instance alkyl or alkoxy dimethicones containing from 16 to 45 carbon atoms, paraffin waxes, ceresins, such as EMW-0003, sold by the company Nippon Seirou, α-olefin oligomers, such as the polymers Performa V® 825, 103 and 260, sold by the company New Phase Technologies; ethylene-propylene copolymers, such as Performalene® EP 700, and mixtures thereof.

A composition according to the invention may comprise one or more non-micronized wax(es) in a content ranging from 0.1% to 30%, for example from 0.2% to 25% or for example from 0.3% to 20% by weight relative to the total weight of the composition.

c) Pasty Compounds

The cosmetic compositions in accordance with the present invention may also comprise at least one pasty compound.

For the purposes of the present invention, the term “pasty” refers to a fatty compound with a reversible solid/liquid change of state and comprising, at a temperature of 25° C., a liquid fraction and a solid fraction.

Polyol esters are for example suitable as pasty compounds according to the invention.

The polyol esters that may be used in the context of the present invention are commercially available or may be prepared in a conventional manner. They are generally of plant origin and may be obtained for example by mono- or polyesterification of a polyol with a C₂-C₃₄ monocarboxylic acid, for instance a fatty acid or with a dicarboxylic acid such as a diacid dimer.

The ester obtained may especially be a polyester, a triester, a diester, a monoester or a mixture thereof. In the present case, the ester may be a mixture of two or more types of ester formed with different carboxylic acids.

In the case of esterification with a monocarboxylic acid, esters with a relatively high molecular weight, ranging from about 200 to 1300 g/mol, may be obtained.

In the esterification reaction with a dicarboxylic acid, a polyol dicarboxylate may be obtained, which has a weight-average molecular weight, determined by gel permeation chromatography (GPC), ranging from 200 to 20 000 g/mol and for example between 2000 and 4000 g/mol.

For the purposes of the present invention, the terms “polyol” and “polyhydric alcohol” should be understood as meaning any organic molecule comprising at least two free hydroxyl groups.

They may for example contain from 2 to 20 carbon atoms, for example from 3 to 10 carbon atoms and for example from 4 to 6 carbon atoms.

The polyol may be chosen, for example, from a diol dimer, glycerol, propylene glycol, butylene glycol, pentylene glycol, hexylene glycol, dipropylene glycol, diethylene glycol, sorbitol, hydroxypropyl sorbitol and 1,2,6-hexanetriol; glycol ethers (especially containing from 3 to 16 carbon atoms) such as mono-, di- or tripropylene glycol (C₁-C₄)alkyl ethers and mono-, di- or triethylene glycol (C₁-C₄)alkyl ethers; and mixtures thereof.

It may also be a “diol dimer”, i.e. saturated diols produced by hydrogenation of the corresponding diacid dimers.

A diol dimer may be produced by hydrogenation of a diacid dimer, which is itself obtained by dimerization of an unsaturated fatty acid for example of C₈ to C₃₄, such as those mentioned previously, for example of C₁₂ to C₂₂ and for example of C₁₆ to C₂₀, for example C₁₈ such as, for example, oleic acid and linoleic acid.

The polyols that are more particularly suitable are sugars chosen from monosaccharides, disaccharides and trisaccharides. Illustrations of these sugars that may for example be mentioned include monosaccharides such as xylose, arabinose, galactose, fructose, mannose and glucose, and mixtures thereof. Illustrations of disaccharide polyols that may more particularly be mentioned include maltose, lactose and sucrose and combinations thereof.

The monocarboxylic acid that may be used in the present invention may contain from 2 to 34 carbon atoms and for example from 10 to 32 carbon atoms.

By way of illustration of monocarboxylic acids that are suitable for the invention, mention may be made of:
saturated linear acids such as butanoic acid, pentanoic acid, hexanoic acid, heptanoic acid, octanoic acid, nonanoic acid, decanoic acid, undecanoic acid, dodecanoic acid, tridecanoic acid, tetradecanoic acid, pentadecanoic acid, hexadecanoic acid, heptadecanoic acid, octadecanoic acid, nonadecanoic acid, eicosanoic acid docosanoic acid and tetracosanoic acid,

branched fatty acids, for instance isobutanoic acid, isopentanoic acid, pivalic acid, isohexanoic acid, isoheptanoic acid, isooctanoic acid, dimethyloctanoic acid, isononanoic acid, isodecanoic acid, isoundecanoic acid, isododecanoic acid, isotridecanoic acid, isotetradecanoic acid, isopentadecanoic acid, isohexadecanoic acid, isoheptadecanoic acid, isooctadecanoic acid, isononadecanoic acid, isoeicosanoic acid, 2-ethylhexanoic acid, 2-butyloctanoic acid, 2-hexyldecanoic acid, 2-octyldodecanoic acid, 2-decyltetradecanoic acid, 2-dodecylhexadecanoic acid, 2-tetradecyloctadecanoic acid, 2-hexadecyloctadecanoic acid and long-chain fatty acids obtained from lanolin,
unsaturated linear C₈ to C₃₄ fatty acids, such as undecenoic acid, linderic acid, myristoleic acid, palmitoleic acid, oleic acid, linoleic acid, elaidinic acid, gadolenoic acid, eicosapentaenoic acid, docosahexaenoic acid, erucic acid, brassidic acid and arachidonic acid,
hydroxy acids such as 2-hydroxybutanoic acid, 2-hydropentanoic acid, 2-hydroxyhexanoic acid, 2-hydroxyheptanoic acid, 2-hydroxyoctanoic acid, 2-hydroxynonanoic acid, 2-hydroxydecanoic acid, 2-hydroxyundecanoic acid, 2-hydroxydodecanoic acid, 2-hydroxytridecanoic acid, 2-hydroxytetradecanoic acid, 2-hydroxyhexadecanoic acid, 2-hydroxyheptadecanoic acid, 2-hydroxyoctadecanoic acid, 12-hydroxyoctadecanoic acid, 2-hydroxynonadecanoic acid, 2-hydroxyeicosanoic acid, 2-hydroxydocosanoic acid and 2-hydroxytetracosanoic acid,
cyclic acids such as cyclohexanoic acid, hydrogenated rosin, rosin, abietic acid, hydrogenated abietic acid, benzoic acid, p-oxybenzoic acid, p-aminobenzoic acid, cinnamic acid, p-methoxycinnamic acid, salicylic acid, gallic acid, pyrrolidonecarboxylic acid and nicotinic acid, and
fatty acids of natural origin, such as the fatty acids of orange oil, of avocado oil, of macadamia oil, of olive oil, of hydrogenated soybean oil, of jojoba oil, of palm oil, of castor oil, of wheatgerm oil, of saffron oil, of cottonseed oil and of mink oil, and mixtures thereof.

In one exemplary embodiment, it may for example be a fatty acid, for example as defined above.

The dicarboxylic acid that may be used according to the invention may contain at least two carboxylic groups per molecule.

It may be represented for example by the formula below:

HOOC—(CH₂)ₙ—COOH

in which n is an integer from 1 to 16 and for example from 3 to 16.

As non-limiting illustrations of dicarboxylic acids that are suitable for the invention, mention may be made for example of malonic acid, succinic acid, glutaric acid, adipic acid, pimelic acid, suberic acid, azelaic acid, sebacic acid, 1,9-nonamethylenedicarboxylic acid, 1,10-decamethylenedicarboxylic acid, 1,11-undecamethylenedicarboxylic acid, 1,12-dodecamethylenedicarboxylic acid, 1,13-tridecamethylenedicarboxylic acid, 1,14-tetradecamethylenedicarboxylic acid, 1,15-pentadecamethylenedicarboxylic acid and 1,16-hexadecamethylenedicarboxylic acid, and mixtures thereof.

The dicarboxylic acid may also be a diacid dimer. The term “diacid dimer” denotes a diacid obtained by polymerization reaction, for example by intermolecular dimerization of at least one unsaturated fatty acid for example of C₈ to C₃₄, such as those mentioned previously, for example of C₁₂ to C₂₂ and for example of C₁₆ to C₂₀, for example of C₁₈ such as, for example, oleic acid and linoleic acid.

Polyol polyesters in which the fatty acid ester units of the polyester comprise saturated or unsaturated chain lengths chosen such that the compound has the required behaviour in terms of pasty compounds according to the invention, are also most particularly suitable as polyol esters.

The unsaturated fatty acid chains are typically branched chains and may for example contain from 12 to about 22 and for example from about 18 to 22 carbon atoms. In one exemplary embodiment, the unsaturated fatty acid chains may be monounsaturated and/or diunsaturated C₁₈ fatty acids.

These long chains may be combined with shorter saturated fatty acid chains. They are generally linear and contain from 2 to about 12, for example from 6 to about 12 and for example from 8 to 12 carbon atoms.

In general, the degree of esterification of these fatty acid esters is such that about 60% of the hydroxyl functions of the polyols and for example about 85% or even 95% of the hydroxyl functions of the polyols are esterified.

As regards the unsaturated long-chain fatty acid ester units, mention may be made for example of lauroleates, myristoleates, palmitoleates, oleates, elaidates, eructates, linoleates, linolenates, arachidonates, eicosapentaenoates and docosahexaenoates. For reasons of stability to oxidation, monounsaturated and diunsaturated fatty acid chains are preferred.

As regards the saturated long-chain unsaturated fatty acid ester units, mention may be made for example of arachidate, behenate, linoserate and serotate esters.

As regards the short-chain saturated fatty acid ester units, they may be for example acetate, caproate, caprylate, caprate and laurate.

As solid polyol fatty acid polyesters that are for example suitable for the invention, mention may be made for example of raffinose octaesters in which the esterifying fatty acid parts are linoleate and behenate, maltose hectaesters in which the esterifying fatty acid parts are derived from sunflower seed oil fatty acid and from lignoserate, sucrose octaesters in which the esterifying fatty acid parts are behenate and oleate, and sucrose octaesters in which the esterifying fatty acid parts are laurates, linoleates and behenates.

Such solid fatty acid polyesters may be obtained according to methods already described for the preparation of the polyol polyesters. In this respect, reference may be made for example to documents U.S. Pat. No. 5,306,516, U.S. Pat. No. 5,306,515, U.S. Pat. No. 5,305,514, U.S. Pat. No. 4,797,300, U.S. Pat. No. 3,963,699, U.S. Pat. No. 4,518,772 and U.S. Pat. No. 4,517,360.

As pasty compounds that may for example be suitable for the formulation of the cosmetic compositions in accordance with the present invention, mention may be made for example of fractionated hydrogenated triglycerides and for example those sold by SIO; hydrogenated plant oils, hydrogenated palm oil, cocoa butter and, for example, those sold by Karlshamns, solid cottonseed oil, for example the oil sold by SIO, and sucrose acetate isobutyrate, for example the product sold by Eastman Chemical.

Among the other pasty compounds that may be used in the composition according to the invention, mention may also be made of lanolines and lanoline derivatives, for instance acetylated lanolines, oxypropylenated lanolines or isopropyl lanolate, and mixtures thereof.

Mention may also be made of pasty silicone compounds such as high molecular weight polydimethylsiloxanes (PDMS) and for example those with pendent chains of the alkyl or alkoxy type containing from 8 to 24 carbon atoms, and a melting point of 20-55° C., for instance stearyl dimethicones, for example those sold by the company Dow Corning under the trade names DC2503® and DC25514®, and mixtures thereof.

A composition according to the invention may comprise one or more pasty compound(s) in a content ranging from 0.1% to 50%, for example from 0.2% to 40% or for example from 0.3% to 30% by weight relative to the total weight of the composition.

The composition according to the invention may also comprise at least one additional pulverulent compound, i.e. a compound other than the microwaxes under consideration according to the invention. The additional pulverulent compound may be chosen from fillers, pulverulent dyestuffs such as pigments and nacres, and mixtures thereof.

The term “fillers” should be understood as meaning white or colourless, mineral or synthetic, lamellar or non-lamellar particles.

These fillers may be chosen from talc, mica, silica, kaolin, poly-β-alanine powder, powders of lauroyllysine, starch, boron nitride, hollow polymer microspheres such as those of polyvinylidene chloride/acrylonitrile, such as Expancel® (Nobel Industrie), acrylic polymer particles, for example of acrylic acid copolymer, for instance Polytrap® (Dow Corning), polyurethane powders, nylon powders, silicone resin microbeads (for example Tospearls® from Toshiba), precipitated calcium carbonate, dicalcium phosphate, magnesium carbonate and magnesium hydrocarbonate, hydroxyapatite, hollow silica microspheres (Silica Beads® from Maprecos), glass or ceramic microcapsules, metal soaps derived from organic carboxylic acids containing from 8 to 22 carbon atoms and for example from 12 to 18 carbon atoms, for example zinc stearate, magnesium stearate or lithium stearate, zinc laurate and magnesium myristate, and mixtures thereof.

These fillers may or may not be surface-treated, especially to make them lipophilic.

These fillers may be present in the composition according to the invention in a content ranging from 0.1% to 30% by weight and for example from 0.1% to 15% by weight relative to the total weight of the composition.

A composition according to the invention may also comprise at least one dyestuff chosen from pulverulent dyestuffs, such as pigments and nacres, and dyes such as lipophilic dyes and hydrophilic dyes, mixtures thereof.

For the purposes of the present invention, the term “pigments” is intended to denote white or colourless, mineral or organic particles that are insoluble in the liquid organic phase, which are intended to colour and/or opacify the composition.

The pigments may be mineral and/or organic pigments. Pigments that may be used include metal oxides, for instance iron oxides (especially yellow, red, brown or black iron oxides), titanium dioxides, cerium oxide, zirconium oxide and chromium oxide; manganese violet, ultramarine blue, Prussian blue, ferric blue, bismuth oxychloride, natural mother-of-pearl, mica coated with titanium or with bismuth oxychloride, coloured nacreous pigments such as titanium mica with iron oxides, titanium mica especially with ferric blue or chromium oxide, titanium mica with an organic pigment of the abovementioned type and nacreous pigments based on bismuth oxychloride, and mixtures thereof.

Iron oxide or titanium dioxide pigments may for example be used in particular.

The pulverulent dyestuffs and especially the pigments may be surface-treated, where appropriate, with a hydrophobic agent to make them compatible with the fatty phase of the composition.

Such hydrophobic-treated pigments are for example described in patent application EP-A-1 086 683.

As illustrative hydrophobic agents that may be used to coat pigments, examples that may be mentioned include aluminium stearoyl glutamate and, for example, isopropyl triisostearyl titanate.

For the purposes of the present invention, the term “nacres” is intended to denote iridescent particles, produced especially by certain molluscs in their shell or else synthesized, which are insoluble in the medium of the composition.

For the purposes of the present invention, the term “dye” is intended to denote compounds, generally organic compounds, which are soluble in the fatty substances such as the oils or in an aqueous and/or aqueous-alcoholic phase.

Among the liposoluble dyes that may be used according to the invention, mention may be made of Sudan Red, D&C Red No. 17, D&C Green No. 6, β-carotene, soybean oil, Sudan Brown, D&C Yellow No. 11, D&C Violet No. 2, D&C Orange No. 5, quinoline yellow, annatto and bromo acids.

The pulverulent dyestuffs may be present in a composition according to the invention in a content ranging from 0.5% to 30% by weight, for example ranging from 5% to 20% by weight and for example ranging from 8% to 15% by weight relative to the total weight of the composition.

The lipophilic and/or hydrophilic dyes may be present in a composition according to the invention in a content ranging from 0.0001% to 10% by weight, for example from 0.01% to 5% by weight and for example from 0.1% to 3% by weight relative to the total weight of the composition.

The composition according to the invention may also contain one or more adjuvants that are common in cosmetics, such as hydrophilic or lipophilic gelling agents and/or thickeners; moisturizers; emollients; hydrophilic or lipophilic active agents; free-radical scavengers; sequestering agents; antioxidants; preserving agents; basifying or acidifying agents; fragrances; and mixtures thereof.

As active agents that may be used in the composition according to the invention, mention may be made, for example, of moisturizers such as protein hydrolysates and polyols, for instance glycerol, glycols, for instance polyethylene glycols, and sugar derivatives; natural extracts; antiinflammatory agents; procyannidol oligomers; vitamins such as vitamin A (retinol), vitamin E (tocopherol), vitamin C (ascorbic acid), vitamin B5 (panthenol), vitamin B3 (niacinamide), derivatives of these vitamins (especially esters) and mixtures thereof; urea; caffeine; salicylic acid and its derivatives; α-hydroxy acids such as lactic acid and glycolic acid and derivatives thereof; retinoids such as carotenoids and vitamin A derivatives; hydrocortisone; melatonin; algal, fungal, plant, yeast or bacterial extracts; enzymes; steroids; antibacterial active agents, for instance 2,4,4′-trichloro-2′-hydroxydiphenyl ether (or triclosan), 3,4,4′-trichlorocarbanilide (or triclocarban) and the acids indicated above, and especially salicylic acid and its derivatives; tensioning agents; and mixtures thereof.

The composition according to the invention may also comprise at least one sunscreen (or UV-screening agent). This agent may be chosen from organic screening agents and physical sunblocks, and mixtures thereof.

As chemical sunscreens that may be used in the composition of the invention, the composition of the invention may comprise any UVA- and UVB-screening agents that may be used in cosmetics.

Needless to say, a person skilled in the art will take care to select the optional adjuvant(s) added to the composition according to the invention and to adjust the amounts thereof such that the advantageous properties intrinsically associated with the composition in accordance with the invention are not, or are not substantially, adversely affected by the envisaged addition.

The examples that follow are given, with no limiting nature, to illustrate the invention.

### EXAMPLE 1

### Liquid Foundation

| | Mass % |
|---|---|
| A1 | |
| Cetyl PEG/PPG-10/1 dimethicone sold under the reference Abil EM 90 | 2.37 |
| by the company Goldschmidt | |
| Polyglyceryl-4 isostearate sold under the reference Isolan GI 34 | 0.39 |
| by the company Goldschmidt | |
| Butyl paraben | 0.08 |
| Tristearine and acetylated ethylene glycol stearate, sold under the reference | 0.39 |
| Unitwix by the company United Guardian | |
| A2 | |
| Cyclopentasiloxane and PEG/PPG-18/18 dimethicone sold under the reference | 2.02 |
| DC 5225C Formulation Aid by the company Dow Corning | |
| A3 | |
| Cyclopentasiloxane sold under the reference DC 245 Fluid by the | 16.90 |
| company Dow Corning | |
| Dimethicone sold under the reference DC Fluid 200 5 cSt by the | 8.40 |
| company Dow Corning | |
| A4 | |
| Synthetic microwax sold under the reference Microease 114S | 16.80 |
| by the company Micro Powders | |
| A5 | |
| Yellow iron oxide coated with aluminium stearoyl glutamate, sold under | 1.55 |
| the reference NAI-C33-8073-10 by the company Myoshi Kasei | |
| Red iron oxide coated with aluminium stearoyl glutamate, sold under | 0.32 |
| the reference NAI-C33-8075-10 by the company Myoshi Kasei | |
| Black iron oxide coated with aluminium stearoyl glutamate, sold under | 0.16 |
| the reference NAI-C33-134-10 by the company Myoshi Kasei | |
| Titanium dioxide coated with aluminium stearoyl glutamate, sold under | 8.87 |
| the reference NAI-C47-051-10 by the company Myoshi Kasei | |
| Iron oxide coated with aluminium stearoyl glutamate, sold under | 0.10 |
| the reference NAI-C33-115-10 by the company Myoshi Kasei | |
| Cyclopentasiloxane, sold under the reference DC 245 Fluid by the | 5.00 |
| company Dow Corning | |
| B1 | |
| Water | 28.98 |
| Methyl paraben | 0.16 |
| Butyleneglycol | 3.96 |
| Magnesium sulfate | 0.55 |
| Propyleneglycol | 2.37 |
| B2 | |
| Hydroxypropyl guar sold under the reference Jaguar HP 105 | 0.08 |
| by the company Rhodia | |
| B3 | |
| Phenoxyethanol | 0.55 |

Procedure

The constituents of phase A1 are weighed out and then placed on a water bath (100° C.). When the mixture is homogeneous, phase A2 is added with stirring (Moritz blender at 2000 rpm), followed by phase A3, at a temperature of 60° C., and finally phase A4 at a temperature of 50° C., with continued stirring.

Phase A5 is prepared separately by grinding the pigments with a three-roll mill, and is then added to the above mixture.

The aqueous phase is prepared by weighing out the constituents of phase B1, which are then brought to the boiling point.

This phase is then stirred (Rayneri blender at a speed of 400-500 rpm) and phase B2 is added thereto. Stirring is continued until a smooth gel is obtained. Phase B3 is then added, at a temperature of about 40° C.

The emulsion is formed at room temperature. The two phases should have a similar temperature (not more than 3° C. difference) close to room temperature.

The aqueous phase is poured into the fatty phase while gradually increasing the stirring speed (Moritz blender up to 4000-4500 rpm). After addition, stirring is continued for 10 minutes.

Result

This foundation is very easy to apply, has good slipperiness and feels very soft and fresh on application.

The resulting makeup is uniform and matt, with no powdery effect. It has a very soft feel and is not tacky.

### EXAMPLE 2

### Liquid Foundation

| | Mass % |
|---|---|
| A1 | |
| Cetyl PEG/PPG-10/1 dimethicone sold under the reference Abil EM 90 | 2.26 |
| by the company Goldschmidt | |
| Polyglyceryl-4 isostearate sold under the reference Isolan GI 34 | 0.38 |
| by the company Goldschmidt | |
| Butyl paraben | 0.07 |
| Tristearine and acetylated ethylene glycol stearate, sold under the reference | 0.38 |
| Unitwix by the company United Guardian | |
| A2 | |
| Cyclopentasiloxane and PEG/PPG-18/18 dimethicone sold under the reference | 1.93 |
| DC 5225C Formulation Aid by the company Dow Corning | |
| A3 | |
| Cyclopentasiloxane sold under the reference DC 245 Fluid by the | 15.93 |
| company Dow Corning | |
| Dimethicone sold under the reference DC Fluid 200 5 cSt by the | 8.03 |
| company Dow Corning | |
| A4 | |
| Synthetic microwax sold under the reference Microease 114S | 20.00 |
| by the company Micro Powders | |
| A5 | |
| Yellow iron oxide coated with aluminium stearoyl glutamate, sold under | 2.70 |
| the reference NAI-C33-8073-10 by the company Myoshi Kasei | |
| Red iron oxide coated with aluminium stearoyl glutamate, sold under | 0.52 |
| the reference NAI-C33-8075-10 by the company Myoshi Kasei | |
| Black iron oxide coated with aluminium stearoyl glutamate, sold under | 0.21 |
| the reference NAI-C33-134-10 by the company Myoshi Kasei | |
| Titanium dioxide coated with aluminium stearoyl glutamate, sold under | 7.45 |
| the reference NAI-C47-051-10 by the company Myoshi Kasei | |
| Iron oxide coated with aluminium stearoyl glutamate, sold under | 0.12 |
| the reference NAI-C33-115-10 by the company Myoshi Kasei | |
| Cyclopentasiloxane, sold under the reference DC 245 Fluid by the | 5.00 |
| company Dow Corning | |
| B1 | |
| Water | 27.70 |
| Methyl paraben | 0.15 |
| Butyleneglycol | 3.78 |
| Magnesium sulfate | 0.53 |
| Propyleneglycol | 2.26 |
| B2 | |
| Hydroxypropyl guar sold under the reference Jaguar HP 105 | 0.07 |
| by the company Rhodia | |
| B3 | |
| Phenoxyethanol | 0.53 |

Procedure

The procedure is the same as that used in Example 1.

This foundation has sensory properties very similar to those of Example 1.

### EXAMPLE 3

### Liquid Foundation

| | Mass % |
|---|---|
| A1 | |
| Cetyl PEG/PPG-10/1 dimethicone sold under the reference Abil EM 90 | 2.37 |
| by the company Goldschmidt | |
| Polyglyceryl-4 isostearate sold under the reference Isolan GI 34 | 0.39 |
| by the company Goldschmidt | |
| Butyl paraben | 0.08 |
| Tristearine and acetylated ethylene glycol stearate, sold under the reference | 0.50 |
| Unitwix by the company United Guardian | |
| A2 | |
| Cyclopentasiloxane and PEG/PPG-18/18 dimethicone sold under the reference | 2.02 |
| DC 5225C Formulation Aid by the company Dow Corning | |
| A3 | |
| Cyclopentasiloxane sold under the reference DC 245 Fluid by the | 15.79 |
| company Dow Corning | |
| Dimethicone sold under the reference DC Fluid 200 5 cSt by the | 8.40 |
| company Dow Corning | |
| A4 | |
| Synthetic microwax sold under the reference Microease 114S | 16.80 |
| by the company Micro Powders | |
| A5 | |
| Yellow iron oxide coated with isopropyl triisostearoyl titanate, sold under | 2.13 |
| the reference BYO-I 2 by the company Kobo | |
| Red iron oxide coated with isopropyl triisostearoyl titanate, sold under | 0.72 |
| the reference BRO-I 2 by the company Kobo | |
| Black iron oxide coated with isopropyl triisostearoyl titanate, sold under | 0.29 |
| the reference BBO-I 2 by the company Kobo | |
| Titanium dioxide coated with isopropyl triisostearoyl titanate, sold under | 7.86 |
| the reference BTD-401 by the company Kobo | |
| Cyclopentasiloxane DC 245 Fluid by the company Dow Corning | 5.00 |
| B1 | |
| Water | 29.98 |
| Methyl paraben | 0.16 |
| Butyleneglycol | 3.96 |
| Magnesium sulfate | 0.55 |
| Propyleneglycol | 2.37 |
| B2 | |
| Hydroxypropyl guar sold under the reference Jaguar HP 105 | 0.08 |
| by the company Rhodia | |
| B3 | |
| Phenoxyethanol | 0.55 |

Procedure

The procedure is the same as that used in Example 1.

This foundation has sensory properties very similar to those of Example 1.

### EXAMPLE 4

### Liquid Foundation

| | Mass % |
|---|---|
| A1 | |
| Cetyl PEG/PPG-10/1 dimethicone sold under the reference Abil EM 90 | 2.26 |
| by the company Goldschmidt | |
| Polyglyceryl-4 isostearate sold under the reference Isolan GI 34 | 0.38 |
| by the company Goldschmidt | |
| Butyl paraben | 0.07 |
| Tristearine and acetylated ethylene glycol stearate, sold under the reference | 0.38 |
| Unitwix by the company United Guardian | |
| A2 | |
| Cyclopentasiloxane and PEG/PPG-18/18 dimethicone sold under the reference | 1.93 |
| DC 5225C Formulation Aid by the company Dow Corning | |
| A3 | |
| Cyclopentasiloxane sold under the reference DC 245 Fluid by the | 15.93 |
| company Dow Corning | |
| Dimethicone sold under the reference DC Fluid 200 5 cSt by the | 8.03 |
| company Dow Corning | |
| A4 | |
| Carnauba microwax sold under the reference Microcare 350 | 20.00 |
| by the company Micro Powders | |
| A5 | |
| Yellow iron oxide coated with aluminium stearoyl glutamate, sold under | 2.7 |
| the reference NAI-C33-8073-10 by the company Myoshi Kasei | |
| Red iron oxide coated with aluminium stearoyl glutamate, sold under | 0.52 |
| the reference NAI-C33-8075-10 by the company Myoshi Kasei | |
| Black iron oxide coated with aluminium stearoyl glutamate, sold under | 0.21 |
| the reference NAI-C33-134-10 by the company Myoshi Kasei | |
| Titanium dioxide coated with aluminium stearoyl glutamate, sold under | 7.45 |
| the reference NAI-C47-051-10 by the company Myoshi Kasei | |
| Iron oxide coated with aluminium stearoyl glutamate, sold under | 0.12 |
| the reference NAI-C33-115-10 by the company Myoshi Kasei | |
| Cyclopentasiloxane, sold under the reference DC 245 Fluid by the | 5.00 |
| company Dow Corning | |
| B1 | |
| Water | 27.70 |
| Methyl paraben | 0.15 |
| Butyleneglycol | 3.78 |
| Magnesium sulfate | 0.53 |
| Propyleneglycol | 2.26 |
| B2 | |
| Hydroxypropyl guar sold under the reference Jaguar HP 105 | 0.07 |
| by the company Rhodia | |
| B3 | |
| Phenoxyethanol | 0.53 |

Procedure

The procedure is the same as that used in Example 1.

The foundation has sensory properties very similar to that of Example 1.

### EXAMPLE 5

### Makeup Base

| | Mass % |
|---|---|
| A1 | |
| Cetyl PEG/PPG-10/1 dimethicone sold under the reference Abil EM 90 | 3.00 |
| by the company Goldschmidt | |
| Polyglyceryl-4 isostearate sold under the reference Isolan GI 34 | 0.50 |
| by the company Goldschmidt | |
| Butyl paraben | 0.10 |
| Tristearine and acetylated ethylene glycol stearate, sold under the reference | 0.50 |
| Unitwix by the company United Guardian | |
| A2 | |
| Cyclopentasiloxane and PEG/PPG-18/18 dimethicone sold under the reference | 2.55 |
| DC 5225C Formulation Aid by the company Dow Corning | |
| A3 | |
| Cyclopentasiloxane sold under the reference DC 245 Fluid by the | 22.00 |
| company Dow Corning | |
| Dimethicone sold under the reference DC Fluid 200 5 cSt by the | 10.00 |
| company Dow Corning | |
| A4 | |
| Synthetic microwax sold under the reference Microease 114S | 15.00 |
| by the company Micro Powders | |
| B1 | |
| Water | 36.65 |
| Methyl paraben | 0.20 |
| Butyleneglycol | 5.00 |
| Magnesium sulfate | 0.70 |
| Propyleneglycol | 3.00 |
| B2 | |
| Hydroxypropyl guar sold under the reference Jaguar HP 105 | 0.10 |
| by the company Rhodia | |
| B3 | |
| Phenoxyethanol | 0.70 |

Procedure

The constituents of phase A1 are weighed out and then placed on a water bath (100° C.). When the mixture is homogeneous, phase A2 is added with stirring (Moritz blender at 2000 rpm), followed by phase A3, at a temperature of 60° C., and finally phase A4 at a temperature of 50° C., with continued stirring.

The aqueous phase is prepared by weighing out the constituents of phase B1, which are then brought to the boiling point. This phase is then stirred (Rayneri blender at a speed of 400-500 rpm) and phase B2 is added thereto. Stirring is maintained until a smooth gel is obtained. Phase B3 is then added, at a temperature of about 40° C.

The emulsion is formed at room temperature. The two phases should have a similar temperature (not more than 3° C. difference) close to room temperature.

The aqueous phase is poured into the fatty phase while gradually increasing the stirring speed (Moritz blender up to 4000-4500 rpm). After addition, stirring is continued for 10 minutes.

Result

This base is very easy to apply, has good slipperiness and feels very soft and fresh on application. The result is uniform and matt, with no powdery effect. It has a very soft feel and is not tacky.

This makeup base may be applied in combination with a foundation or a loose or compacted powder.

Although the present invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims.

1. Non-compacted cosmetic composition for caring for and/or making up keratin materials, comprising, in a physiologically acceptable medium, at least 22% by weight of water relative to the total weight of the composition and at least one fatty phase comprising at least one dispersion of micronized wax particles, the said particles being present in a content of between 5% and 50% by weight relative to the total weight of the composition.

2. Composition according to claim 1, comprising at least 25% by weight of water relative to the total weight of the composition.

3. Composition according to claim 1, wherein the micronized wax particles are present in a content of between 10% and 30% by weight relative to the total weight of the composition.

4. Composition according to claim 1, wherein the micronized wax particles have a number-average size of less than or equal to 50 μm.

5. Composition according to claim 1, wherein the micronized wax is chosen from the group consisting of hydrocarbon-based, silicone and/or fluoro micronized waxes, optionally comprising ester, amide, acid or hydroxyl functions.

6. Composition according to claim 1, wherein the micronized wax is chosen from the group consisting of optionally modified beeswax, carnauba wax, candelilla wax, paraffin waxes, microcristalline waxes, montan wax, hydrogenated oils, waxes obtained by Fischer-Tropsch synthesis, fatty acid esters and amides, silicone waxes, containing from 10 to 45 carbon atoms, and fatty acids, and mixtures thereof.

7. Composition according to claim 1, also comprising at least one compound chosen from oils, non-micronized waxes and pasty fatty substances, and mixtures thereof.

8. Composition according to claim 7, wherein the oils are chosen from volatile or non-volatile hydrocarbon-based oils of animal, plant or mineral origin, synthetic oils, silicone oils and fluoro oils, and mixtures thereof.

9. Composition according to claim 1, which is in a fluid form.

10. Composition according to claim 1, which is in the form of an oil-in-water or water-in-oil simple emulsion, or a multiple emulsion.

11. Composition according to claim 10, which is in the form of a water-in-oil emulsion.

12. Composition according to claim 1, also comprising at least one dyestuff chosen from pulverulent dyestuffs, dyes, and mixtures thereof.

13. Composition according to claim 12, wherein the pulverulent dyestuffs are chosen from pigments and nacres.

14. Composition according to claim 12, wherein the dyes are chosen from lipophilic dyes and hydrophilic dyes.

15. Composition according to claim 12, wherein the pulverulent dyestuffs are surface-treated with a hydrophobic agent.

16. Composition according to claim 15, wherein the hydrophobic agent is chosen from aluminium stearoyl glutamate and isopropyl triisostearoyl titanate.

17. Composition according to claim 12, wherein the pulverulent dyestuffs are present in a content ranging from 0.5% to 30% by weight relative to the total weight of the composition.

18. Composition according to claim 14, wherein the lipophilic and/or hydrophilic dyes are present in a content ranging from 0.0001% to 10% by weight relative to the total weight of the composition.

19. Composition according to claim 1, which is intended to be applied to the skin and/or the lips.

20. Composition according to claim 1, which is in the form of a paste or a fluid cream.

21. Composition according to claim 20, which is in the form of a fluid foundation or a fluid skin makeup base.
